(19) United States
(12) Patent Application Publication
(10) Pub. No.: US 2022/0126079 A1
(43) Pub. Date: Apr. 28, 2022

(54) Applicator for Microneedle Patch

(71) Applicant: LTS Lohmann Therapie-Systeme AG, Andemach (DE)

(72) Inventors: Markus Bee, Kettig (DE); Ronald Hackbarth, Koblenz (DE)

(21) Appl. No.: 17/432,581

(22) PCT Filed: Feb. 19, 2020
(86) PCT No.: PCT/EP2020/054362

§ 371 Date: Aug. 20, 2021

(30) Foreign Application Priority Data
Feb. 21, 2019 (DE) 10 2019 001 251.8

(51) Int. Cl. A61M 37/00; A61K 9/00

(52) U.S. Cl. CPC A61M 37/0015; A61K 9/0021; A61M 2037/0061; A61M 2037/0046; A61M 2037/0023

(57) ABSTRACT

An applicator comprising a handle piece and a pressing body for a microneedle patch, which pressing body is connected to the handle piece. A unit comprising an applicator of this type and comprising a microneedle patch having a multiplicity of microneedles. The pressing body is mounted for movement relative to the handle piece in a sliding joint or in a pivot joint at least between an idle position and a usage position by means of external operating forces and an internal restoring device. The pressing body has a pressing surface which is spaced apart from the pivot joint or sliding joint. In the idle position, the pressing body is not loaded by external operating forces, and the restoring device has the lowest internal energy. The pressing body can be moved relative to the handle piece toward the usage position by means of external operating forces acting on the pressing surface.

### CROSS-REFERENCE TO RELATED APPLICATIONS

This application is the United States national phase of International Application No. PCT/EP2020/054362 filed Feb. 19, 2020, and claims priority to German Patent Application No. 102019001251.8 filed Feb. 21, 2019, the disclosures of which are hereby incorporated by reference in their entirety.

### BACKGROUND OF THE INVENTION

### Field of the Invention

The invention relates to an applicator having a handle piece and having a pressing body for a microneedle patch, which pressing body is connected to the handle piece, and a unit comprising an applicator of this type and comprising a microneedle patch having a plurality of microneedles.

### Description of Related Art

From US 2018/0177991 A1, an applicator for a microneedle patch is known. The needles are pressed into the patient's skin by means of the varying normal force component in a rolling action of the presser.

The object underlying the present invention is that of enabling the microneedles to be pressed in substantially uniformly.

### SUMMARY OF THE INVENTION

This problem is solved by the features of the main claim. For this purpose, the pressing body is mounted for movement relative to the handle piece in a sliding joint or in a pivot joint at least between an idle position and a usage position by means of external operating forces and an internal restoring device. The pressing body has a pressing surface spaced apart from the pivot joint or sliding joint. In the idle position, the pressing body is not loaded by external operating forces and the restoring device has the lowest internal energy. Furthermore, the pressing body is movable relative to the handle piece towards the usage position by means of external operating forces acting on the pressing surface, the internal energy of the restoring device thus being increased.

In the unit comprising the applicator and the microneedle patch, the smallest radius of the pressing surface is at least one and a half times the product of the circle constant 7 and the length of the heel of a needle in the patch's longitudinal direction. In a conical microneedle, for example, this length is the diameter of the heel of the needle. In a pyramidal microneedle, this length corresponds e.g. to the edge length or the diagonal of the heel of the needle, depending on the arrangement of the microneedle relative to the patch's longitudinal direction. Other shapes of the microneedles are also possible.

The pressing body is mounted pivotably or longitudinally slidably relative to the handle piece. In the idle position, the lowest forces act on the pressing body and on the sliding or pivot joint. The restoring device is free of load.

As soon as the operator presses the applicator on to the microneedle patch and/or e.g. draws the applicator along the microneedle patch, an external operating force acts on the pressing surface. The pressing body is pivoted or slid relative to the handle piece towards the usage position, thus loading the restoring device. The restoring force generated when the restoring device is loaded correlates with the force applied by the operator and with the pressing force of the applicator on the microneedle patch. Thus, when the applicator is drawn or pushed along the microneedle patch, the microneedles are pressed uniformly into the skin and secured with the aid of an overpatch.

A sliding body or a rolling body can be employed as the pressing body. The pressing body has a curved pressing surface. The curvature is of a continuous configuration. It can have a constant radius or can be composed of a plurality of regions of different radii. All the radius center lines of the pressing surface lie parallel to one another. For example, each radius center line lies in a plane normal to a longitudinal direction of the handle piece. At least in the usage position, at least the radius of curvature center line with the smallest radius of curvature is skew to the longitudinal axis of the handle piece. The radius of curvature center line therefore has no point of intersection with the longitudinal axis.

The smallest radius of the pressing surface is selected according to the geometry of the microneedles of the microneedle patch. In this way, it is possible to prevent canting of the microneedles upon introduction into the skin.

Further details of the invention can be taken from the subclaims and from the following descriptions of schematically illustrated exemplary embodiments.

### BRIEF DESCRIPTION OF THE DRAWINGS

FIG. 1: applicator in the idle position;

FIG. 2: applicator in the usage position;

FIG. 3: rear view of the applicator;

FIG. 4: applicator fixing the microneedle patch on the skin;

FIG. 5: unit comprising applicator and microneedle patch;

FIG. 6: section of the pressing body of the unit from FIG. 5;

FIG. 7: unit from FIG. 5 during application of the microneedle patch;

FIG. 8: detail of the microneedle patch with the cover film;

FIG. 9: applicator with sliding joint;

FIG. 10: applicator with dispensing device in the ready position;

FIG. 11: applicator from FIG. 10 during application;

FIG. 12: applicator from FIG. 10 after applying the microneedle patch.

### DETAILED DESCRIPTION

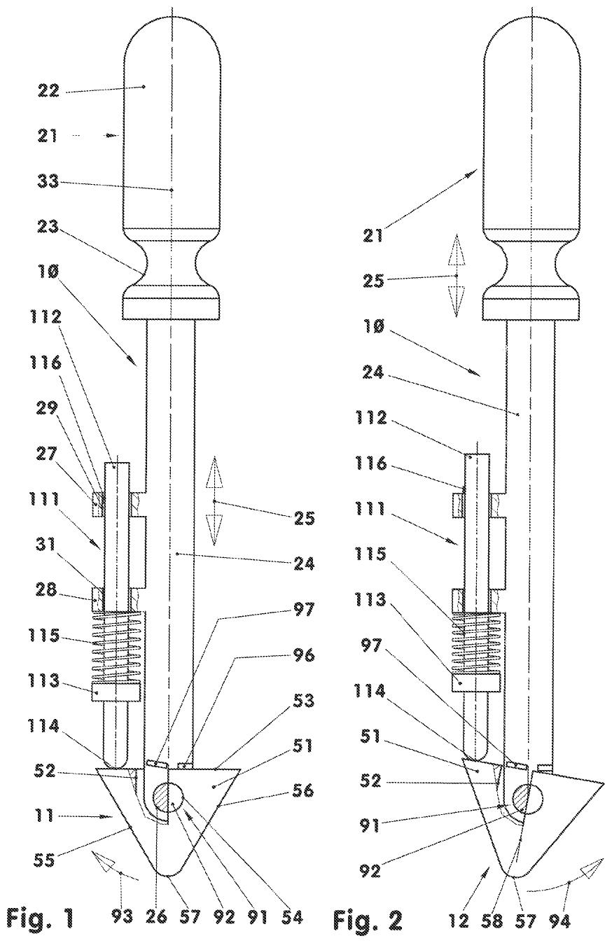

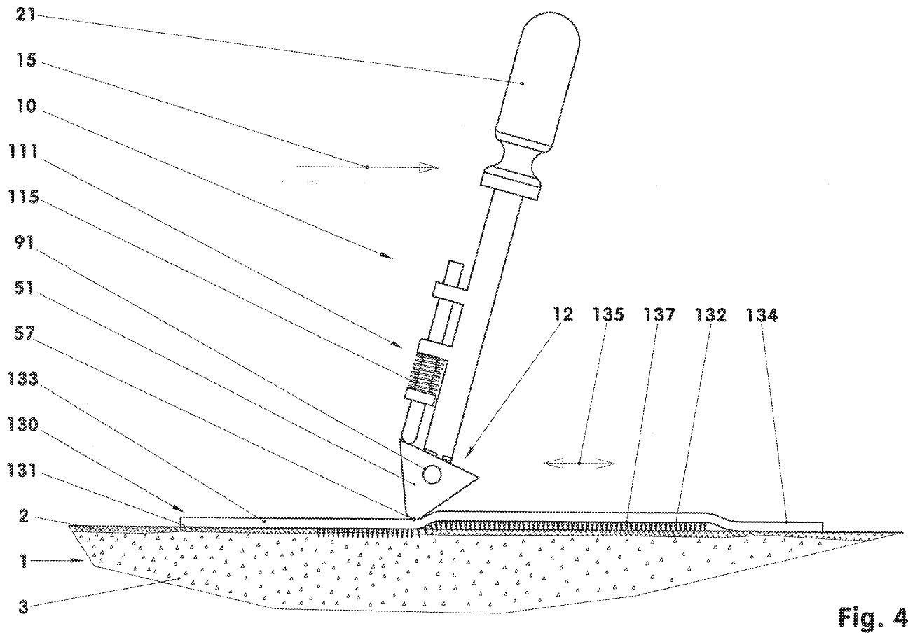

FIG. 1 is a greatly simplified illustration of an applicator (10) in an idle position (11). FIG. 2 shows this applicator (10) in a usage position (12). Applicators (10) of this type are employed to fix microneedle patches (130) on the skin (1) of a patient, see FIG. 4. The individual microneedle patch (130) has a plurality of e.g. geometrically identically configured microneedles (132) on the bottom side (131) facing away from the applicator (10). These microneedles (132) are held together by a backing layer, not illustrated here, which is covered by an overpatch (133). During application, the microneedles (132) are pushed or pressed into the skin (1) with the aid of the applicator (10). After the microneedle patch (130) has been secured on the skin (1), for example active substances contained in the microneedle patch (130) can be delivered into layers of skin (3) beneath the stratum corneum (2).

The overpatch (133), see FIG. 4, projects beyond the region of the microneedles (132) in the patch's longitudinal direction (135) and in the patch's transverse direction, oriented normal thereto. In the state as supplied, see e.g. FIG. 8, the overpatch is covered by means of a sterile barrier (not illustrated here) and a cover film (136), e.g. a release liner. Both the sterile barrier and the cover film (136) have to be removed before applying the microneedle patch (130).

The applicator (10) illustrated in FIGS. 1-4 comprises a handle piece (21) and a pressing body (51) connected thereto. In all the illustrations, the handle piece (21) is shown scaled down compared to the pressing body (51) and the microneedle patch (130). The connection of the pressing body (51) to the handle piece (21) in the exemplary embodiment is in the form of a pivot joint (91) with a restoring device (111).

The handle piece (21) has a ball-of-the-thumb body (22) and a finger recess (23). This region of the handle piece (21) is configured e.g. with rotational symmetry in relation to a longitudinal axis (33) of the handle piece (21). The finger recess (23) in this exemplary embodiment is a circumferential channel. Furthermore, the handle piece (21) has a support bar (24). In the exemplary embodiment, this has a substantially square cross-sectional area. The center line of the support bar (24) lies e.g. in the longitudinal axis (33) of the handle piece (21). At its end facing away from the ball-of-the-thumb body (22), the support bar (24) has a swivel pin receptacle (26). In the exemplary embodiment, a swivel pin (92) is mounted in this swivel pin receptacle (26) and passes through the pressing body (51). The pivot axis (95) forming the center line of the swivel pin receptacle (26) is oriented e.g. normal to the longitudinal axis (33) of the handle piece (21) in the idle position (11). The pivot axis (95) can also be aligned skew to the longitudinal axis (33) in the idle position (11). The pressing body (51) is thus pivotably mounted relative to the handle piece (21) by means of a pivot joint (91). The pivot joint (91) has two degrees of freedom (93, 94) oriented in opposite directions. The two pivoting degrees of freedom (93, 94) are limited in this exemplary embodiment by means of stops (96, 97). In the illustration of FIG. 1, a first stop (96) limits the pivot range of the pressing body (51) relative to the handle piece (21) in the idle position (11). This first stop (96) forms a securing element (96) of the applicator (10) in the idle position (11). According to FIG. 2, the pivot range of the pressing body (51) relative to the handle piece (21) is limited by means of the second stop (97) in the usage position (12). The applicator (10) can also be configured without the second stop (97). Both the first stop (96) and the second stop (97) can also be arranged differently.

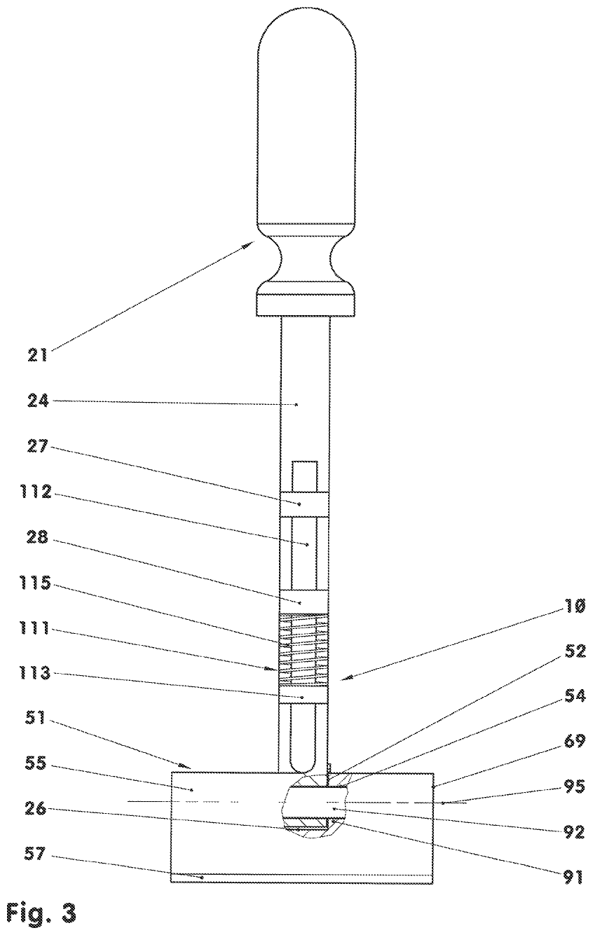

The pressing body (51) is constructed e.g. in a prism shape. In the exemplary embodiment, its cross-sectional area oriented normal to the pivot axis (95) corresponds at least approximately to the area of an equilateral triangle. The cross-sectional area can also have more than three corners or rounded angles. It can also be configured asymmetrically. In the central region of the pivot axis (95), see FIG. 3, the pressing body (51) has a handle piece receptacle (52). The handle piece (21) is mounted in this handle piece receptacle (52), which is in the form of a cut-out. It is also possible for the handle piece (21) to grip the pressing body (51) at the two end faces thereof, which are e.g. parallel to each other. In the exemplary embodiment, the pressing body (51) has e.g. a planar upper side (53). In the idle position (11), the first stop (96) abuts against this upper side (53), and in the usage position (12) the second stop (97) abuts against this upper side (53). In the illustrations of FIGS. 1 and 2, the swivel pin recess (54) of the pressing piece (51) is offset towards the upper side (53) relative to the geometric center line of the cross-sectional area of the pressing body (51).

The upper side (53) in the exemplary embodiment is delimited by two flanks (55, 56). These are e.g. planar surfaces, which are connected to each other by means of a pressing surface (57) facing away from the upper side (53). The pressing surface (57) in this exemplary embodiment is a monoaxially curved surface. The center line of the curvature passes through the pressing body (51) parallel to the pivot axis (95). The radius of curvature in this exemplary embodiment is 5 millimeters. The pressing surface (57) can also be composed of a plurality of curved portions. The center lines of these curved portions, which cover the entire width of the pressing body (51) for example lie parallel to one another in this case. The transitions between the individual curved portions are configured continuously. The transitions from the pressing surface (57) to the flanks (55, 56) can also be configured continuously.

On the support bar (24) in the exemplary embodiment, a mount (27, 28) for the restoring device (111) is arranged. This mount (27, 28) comprises e.g. two guide tabs (27, 28) secured on the support bar (24). These guide tabs (27, 28) can also be parts of the support bar (24). They are spaced apart from one another in the longitudinal direction (25) of the handle piece (21). In the exemplary embodiment, the distance between the guide tabs (27, 28) is 15% of the length of the support bar (24). Each of the guide tabs (27; 28) has a through-opening (29; 31). The two through-openings (29, 31) are, for example, flush with one another. The cross-sectional area of the individual through-opening (29; 31) can be circular, rectangular, hexagonal, etc.

The restoring device (111) comprises a pressing bar (112) mounted in the guide tabs (27, 28) and a spring energy store (115) seated on the pressing bar (112). The pressing bar (112) inserted in the through-openings (29, 31) lies parallel to the longitudinal direction (25) of the handle piece (21). Its upper end protrudes out of the upper guide tab (27). Below the lower guide tab (28), the pressing bar (112) has a supporting collar (113). This has the shape of e.g. a circumferential annular collar. The lower pressing end (114) of the pressing bar (112) contacting the pressing body (51) in FIGS. 1 and 2 is hemispherical in the exemplary embodiment.

In the upper region, the pressing bar (112) has an indicator (116), e.g. a marking (116). This is e.g. a colored ring, a dot, a notch, etc. In the illustration of FIG. 1, this marking (116) lies within the upper guide tab (27). In the usage position (12) shown in FIG. 2, the marking (116) lies above the upper guide tab (27). Instead of an individual marking (116), it is possible e.g. to apply markings of different colors arranged one below the other on the pressing bar (112). For example, the top marking could be red, a middle marking yellow and the bottom marking (116) green. Instead of the visual signal by means of the marking (116) as described, the applicator (10) can also provide the operator with a haptic, acoustic, etc. signal.

With the supporting collar (113), the pressing bar (112) supports the spring energy store (115). In the exemplary embodiment, this is in the form of a compression spring (115). The compression spring (115) in the illustration of FIG. 1 is seated between the supporting collar (113) and the lower guide tab (28), pretensioned to a residual energy value. In this exemplary embodiment, in the idle position (11) this impressed force of the restoring device (111) presses the pressing bar (112) on to the pressing body (51) abutting the securing element (96).

The spring energy store (115) can also be fixed on the supporting collar (113) and on the lower guide tab (28) e.g. in a form-fitting manner. Furthermore, the pressing bar (112) can be connected with its pressing end (114) to the pressing body (51) e.g. by means of a sliding pivot joint. In embodiments of this type, it is possible for example to omit the stops (96, 97).

When the pressing body (51) moves out of the idle position (11) towards the usage position (12), the pressing body (51) is pivoted about the pivot axis (95). The upper side (53) of the pressing body (51) presses on the pressing end (114) of the pressing bar (112). The pressing bar (112) is displaced upwards. As this takes place, the supporting collar (113) and the lower guide tab (28) compress the compression spring (115) e.g. without hysteresis. The internal energy of the restoring device (111) increases. At the same time, the marking (116) migrates upwards such that e.g. the green region becomes visible above the upper guide tab (27). When pressure is removed from the pressing body (51), it springs back to the idle position (11) again, unloading the spring energy store (115).

The restoring device (111) can also comprise a tension spring, a coil torsion spring, a conical coil compression spring, a disc spring assembly, etc. as the spring energy store (115). All these spring energy stores (115) are configured such that the restoring device (111) has the lowest internal energy in the idle position (11). The spring energy store (115) in this case can be completely free of load or can have a slight pre-tension. When a spring energy store (115) with pre-tension is employed, the pressing body (51) is constantly loaded towards the idle position (11) relative to the handle piece (21). When the pressing body (51) moves towards the usage position (12), the relevant spring energy store (115) undergoes additional loading. Its internal energy is increased. Thus, the restoring force also rises.

If, for example, a coil torsion spring or leg spring is employed, this engages around the pivot axis (95) or the swivel pin (92). For example, one leg of the leg spring is fixed, e.g. clamped, on the pressing body (51) and the other leg on the handle piece (21). In the idle position (11), for example, the leg spring is not loaded. The position of the pressing body (51) relative to the handle piece (21) is retained even without the first stop (96). As soon as the pressing body (51) is pivoted out of this position, the legs of the leg spring are pivoted relative to one another about the pivot axis (95). The leg spring is loaded, the internal energy of the restoring device (111) thus being increased. The pressing body (51) can be configured as described above or e.g. as a roller. In this exemplary embodiment, an indicator (116) can indicate the angular orientation of the pressing body (51) relative to the handle piece (21), for example.

Before the applicator (10) illustrated in FIGS. 1 to 4 is employed, the microneedle patch (130) is placed on the patient's skin (1). After the cover film (136) has been removed, the applicator (10) is drawn over the microneedle patch (130) in the patch's longitudinal direction (135), for example, see FIG. 4. The operator positions the applicator (10) obliquely while drawing it. He presses it against the microneedle patch (130) in such a way that the back edge of the overpatch (133) in the patch's longitudinal direction (135) is pressed on to the patient's skin (1) first. The width of the pressing body (51) in the patch's transverse direction is greater than or equal to the width of the microneedle patch (130). The microneedle patch (130) is fixed on the skin (1) over the entire width in the patch's transverse direction (135). In this way, air inclusions, for example, are minimized.

When the applicator (10) is drawn and the pressing body (51) is pressed on to the microneedle patch (130), the pressing body (51) slides with its pressing surface (57) along the surface (134) of the overpatch (133). An external operating force acts on the pressing surface (57). This external operating force is oriented against the drawing direction (15) of the applicator (10). At least in the region of the overpatch (133), the external operating force is also oriented parallel to the surface (134) of the microneedle patch (130). The external operating force thus delays the movement of the applicator (10). The pressing body (51) is pivoted about the pivot axis (95) of the pivot joint (91) towards the usage position (12). The spring energy store (115) is loaded as a function of the amount of the pressing force of the pressing body (51) on the microneedle patch (130). For example, the green marking (116) becomes visible. This signal signifies for the operator e.g. that the pressing force is sufficiently high. The operator can continue to secure the microneedle patch (130). The pressing body (51) is now in the usage position (12) relative to the handle piece (21), or in a region adjacent to the usage position (12).

As the applicator (10) continues to be drawn slowly along the microneedle patch (130), the individual microneedles (132) of the microneedle patch (130) are pressed into the skin (1). For example, the radius of the pressing surface (57) is at least 1.5 times the product of the circle constant π and the diameter of the heel of a needle (137). In the exemplary embodiment, the pressing in of the microneedles (132) takes place in rows. However, it is also possible to draw the applicator (10) e.g. diagonally over the microneedle patch (130). The operator can continue to monitor the pressing force by means of the visual, haptic or acoustic indications. If the pressing force becomes too low, i.e. if for example the green marking (116) ceases to be visible, he can increase the pressing force again by pressing harder on the handle piece (21).

When the microneedle patch (130) is fixed by means of the applicator (10), the overpatch (133) is also secured on the patient's skin (1). After the application, the applicator (10) can be either reused or disposed of. When the applicator (10) is removed from the microneedle patch (130) and from the patient's skin (1), the load is released from the compressed spring energy store (115). The compression spring (115) elongates. By means of the released energy of the restoring device (111), the pressing body (51) is returned to the idle position (11) relative to the handle piece (21).

The use of the applicator (10) with a differently constructed restoring device (111) takes place similarly. When the applicator (10) is loaded, the force applied by the operator is divided relative to the microneedle patch (130) into a tangential component along the microneedle patch (130) and a normal component towards the microneedle patch (130). Each one of these force components, and thus also the resultant of these components, causes a pivoting of the pressing body (51) relative to the handle piece (21) towards the usage position (12). For example, the pivot angle achieved can also be monitored by means of an indicator in this exemplary embodiment. This ensures a minimum pressing force for the reliable insertion of the microneedles (132). When a pressing body (51) in the form of a roller is employed, this can roll over and/or slide along the microneedle patch (130).

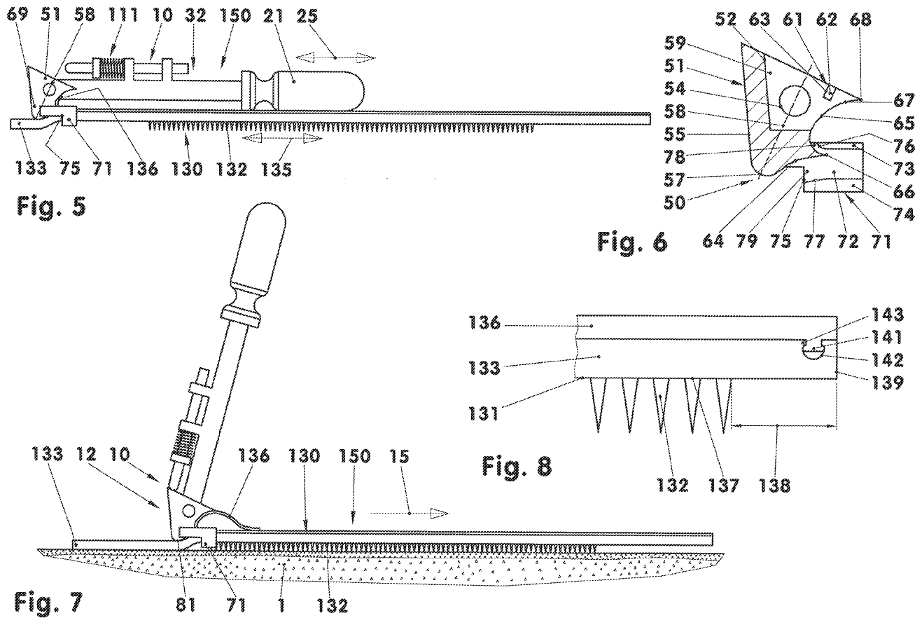

FIGS. 5-7 show a unit (150) composed of an applicator (10) and a microneedle patch (130), and the pressing body (51) as an individual part. The unit (150) is supplied e.g. for single use. In this unit (150), the microneedle patch (130) carries the cover film (136) on the overpatch (133). In the state as supplied, illustrated in FIG. 5, the applicator (10), of which the handle piece (21) lies on the cover film (136), is arranged on the edge of the overpatch (133) and the cover film (136).

The applicator (10) in this exemplary embodiment is substantially constructed as described in connection with the exemplary embodiment illustrated in FIGS. 1-4. The spring energy store (115) is connected e.g. in a form-fitting manner both to the supporting collar (113) and to the lower guide tab (28). The handle piece (21) is in a distribution position (32) in which its longitudinal direction (25) forms an angle of e.g. 65 degrees with a connection plane (58) spanned by the pivot axis (95) and the radius center line of the pressing surface (57). The same angle is formed e.g. by the connection plane (58) in this illustration with the patch's longitudinal direction (135). The ratio of the radius of the pressing surface (57) to the diameter of the heel of a needle (137) or to the length of the heel of a needle (137) in the patch's longitudinal direction (135) corresponds in this exemplary embodiment to the above-mentioned ratio.

In FIG. 6, a longitudinal section of the pressing body (51) of this applicator (10) is illustrated. The section plane of this illustration lies in the vertical central longitudinal plane of the applicator (10). The swivel pin recess (54), the pressing surface (57), the rear flank (55) and the upper side (53) are configured as described in conjunction with the first exemplary embodiment. The handle piece receptacle (52) is open towards the front flank (56). Thus, the handle piece (21) is mounted e.g. without a stop in the distribution position (32).

In the handle piece receptacle (52) in this exemplary embodiment, a locking mechanism or wedge catch (61) is arranged on at least one recess surface (59) oriented parallel to a vertical central longitudinal plane. This comprises e.g. an elastically deformable wedge (62), the thickness of which increases from the front flank (56) towards the rear flank (55). The wedge surface (63) pointing towards the connection plane (58) is a locking surface. When the handle piece (21) pivots from the distribution position (32) towards the idle position (11), the support bar (24) locks behind the wedge surface (63) in the idle position (11). The locking mechanism or wedge catch (61) thus e.g. non-releasably blocks the handle piece (21) from pivoting back to the distribution position (32). In the idle position (11), the locking mechanism or wedge catch (61) thus forms a securing element (61). The applicator (10) illustrated in FIGS. 5-7 can also be configured without the locking mechanism or wedge catch (61).

The front flank (56) in this exemplary embodiment comprises two guiding surfaces (64, 65), which are separated from one another by a separating edge (66). The separating edge (66) runs parallel to the pivot axis (95). It protrudes from the pressing body (51) at an acute angle in the drawing direction (15). In the separating edge (66), the two guiding surfaces (64, 65), which are configured with a concave curvature, form an angle of 10 degrees to 20 degrees, for example. In the exemplary embodiment, the angle formed is 17 degrees. The plane of the bisector of the separating edge (66) forms the same angle with the connection plane (58) as the patch's longitudinal direction (135) with the connection plane (58).

In the exemplary embodiment, the two guiding surfaces (64, 65) are composed of a plurality of monoaxial curved portions lying parallel to one another. The pressing body (51) has the same cross-sectional area over its entire width, with the exception of the handle piece receptacle (52). The imaginary center lines of the individual curved portions of the two guiding surfaces (64, 65) lie parallel to the pivot axis (95). These center lines are offset relative to the pressing body (51) in the drawing direction (15). The lower guiding surface (64) transitions continuously into the pressing surface (57). For example, its radius of curvature decreases from the separating edge (66). The lower guiding surface (64) then transitions tangentially into the pressing surface (57) having an opposite curvature.

The upper guiding surface (65) in the illustration of FIG. 6 has a radius of curvature that widens helically from the separating edge (66). In the exemplary embodiment, the tangential plane is parallel to the plane of the bisector of the separating edge (66) on the upper runout (67) of the upper guiding surface (65). The upper guiding surface (65) in the exemplary embodiment meets the upper side (53) of the pressing body (51) in a longitudinal edge (68).

On each of the end faces (69), the pressing body (51) has a guide element (71). The pressing body (51) and the two guide elements (71) in this exemplary embodiment form a pressing body group (50). In this pressing body group (50), the two guide elements (71), which are configured as mirror images of each other, are rigidly connected to the pressing body (51). For example, they are molded on. However, it is also possible to connect each of the guide elements (71) to the pressing body (51) in pivot joints. In the case of pivotable guide elements (71), the pivot axes of the two guide elements (71), which are in alignment with one another, lie parallel to the radius center line of the pressing surface (57).

The individual guide element (71) has a guide plate (72) and two guide webs (73, 74) each pointing towards the vertical central longitudinal plane of the pressing body (51). The guide plates (72) lie parallel to the vertical central longitudinal plane on the outer sides of the pressing body (51). They have e.g. a square enveloping contour. At their lower end, pointing towards the pressing surface (57), they have a notch (75).

The guide webs (73, 74) are configured so as to be flat. At their end pointing towards the pressing surface (57) they taper in a curved manner from inside to outside. The center axis of the respective radius corresponds e.g. to the center axis of the curved portion of the respective adjacent guiding surface (64; 65). The distance of the upper taper (76) from the upper guiding surface (65) corresponds to the thickness of the cover film (136). The distance of the lower taper (77) from the lower guiding surface (64) corresponds to the thickness of the overpatch (133). The two guide webs (73, 74) protrude e.g. by the amount of the lateral projection (138) of the overpatch (133) beyond the microneedles (132), see FIG. 8, towards the vertical central longitudinal plane. It is also possible, however, for the upper guide web (73) to be configured continuously between the two guide plates (72).

FIG. 8 shows a detail of the microneedle patch (130) with the cover film (136). For example, the right-hand edge of the microneedle patch (130) is illustrated, as seen in the patch's longitudinal direction (135). On both longitudinal sides (139), the cover film (136) is connected in a form-fitting manner to the microneedle patch (130). In this case, for example, the cover film (136) has an elastically deformable longitudinal lug (141), which engages in an elastically deformable longitudinal groove (142) of the microneedle patch (130) with a gripping element (143).

In the unit (150) illustrated in FIGS. 5 and 7, the applicator (10) engages with the separating edge (66) into the interspace between the cover film (136) and the overpatch (133). The upper guide web (73) lies on the cover film (136). The lower guide web (74) is in contact with the bottom side (131) of the overpatch (133).

The cover film (136) is introduced with a free end into an upper guide path (78) between the separating edge (66) and the upper guide web (73) and between the upper taper (76) and the upper guiding surface (65). For example, the cover film (136) already abuts against the upper guiding surface (65).

The overpatch (133) is guided along the lower guide path (79). This runs between the separating edge (66) and the lower guide web (74) and between the lower taper (77) and the lower guiding surface (64). In the exemplary embodiment, the overpatch (133) is guided to below the pressing surface (57) in the distribution position (32) illustrated in FIG. 5.

To use the microneedle patch (130), it is stuck on to the patient's skin (1) e.g. with the end of the overpatch (133) where the applicator (10) is located. The handle piece (21) is folded out of the distribution position (32) illustrated in FIG. 5 until it actuates the locking mechanism or wedge catch (61), for example. The pressing body (51) is now in the idle position (11) relative to the handle piece (21). The spring energy store (115) is not loaded or is loaded only slightly.

The operator now draws the applicator (10) along the microneedle patch (130) in the drawing direction (15), pressing it at the same time. The applicator (10) is pressed in the longitudinal direction (25) of the handle piece (21). Since the longitudinal direction (25) forms an angle not equal to 0 degrees or 180 degrees with the connection plane (58) during the drawing operation, a torque acts on the pressing body (51) around the current polar line (81). This current polar line (81) is the contact line of the pressing surface (57) with the overpatch (133). The pressing surface (57) rolls over the overpatch (133) when the pressing body (51) is displaced, further increasing the angle between the longitudinal axis (33) and the connection plane (58). The spring energy store (115) is loaded until an equilibrium is obtained between the spring force and the force applied by the operator. At the same time, for example, the marking (116) migrates into the visible region.

When the applicator (10) is drawn along the microneedle patch (130), the separating edge (66) separates the connection between the cover film (136) and the overpatch (133). The cover film (136) is guided off along the upper guiding surface (65) and for example settles loosely on the as yet unprocessed region of the cover film (136).

The microneedle patch (130) with the overpatch (133) is guided downwards to the pressing surface (57) by means of the separating edge (66). The microneedles (132) are pressed into the skin (1) e.g. in rows. The overpatch (133) is pressed on to the skin (1) and sticks there by adhesion.

As soon as the microneedle patch (130) is fixed on the patient's skin (1), the cover film (136) is removed completely. The applicator (10) has become released from the microneedle patch (130).

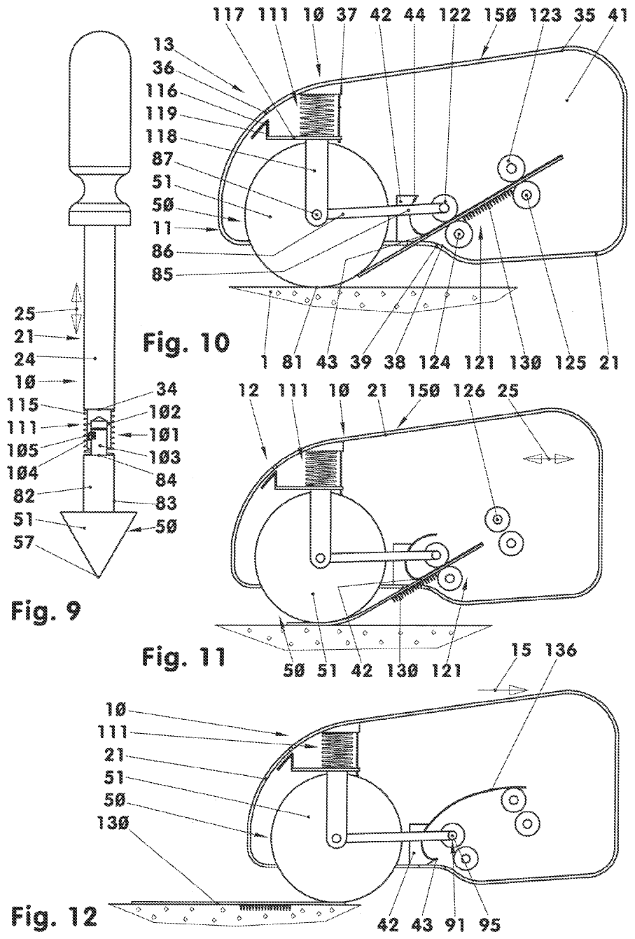

FIG. 9 shows an applicator (10) with a sliding joint (101). This sliding joint (101) connects a pressing body group (50) to the handle piece (21). The sliding joint (101) consists of a hollow prism (102), in which a solid prism (103) is guided telescopically. A restoring device (111), which is supported on the handle piece (21) and on the pressing body group (50), is seated on the sliding joint (101).

The handle piece (21) is substantially constructed as described in connection with the first exemplary embodiment. The support bar (24) has a region with a circular cross-section, which terminates at e.g. a circumferential retaining collar (34). The hollow prism (102) is centrally molded on to this retaining collar (34). This hollow prism is e.g. a hollow bar with a circular cross-sectional area. In the exemplary embodiment, this hollow prism (102) has a longitudinal slot (104) in the manner of an elongated hole oriented in the longitudinal direction (25).

The support bar (24) can also have a different, e.g. a square, cross-sectional area. The retaining collar (34) can consist of individual segments or portions. The hollow prism (102) can also have e.g. a square or other cross-sectional area that deviates from a circular cross-sectional area.

The pressing body group (50) comprises a pressing body (51) and a guide bar (82) arranged on the pressing body (51). The pressing body (51) is e.g. substantially constructed as described in connection with the first exemplary embodiment. The guide bar (82) is rigidly connected to the pressing body (51). It protrudes therefrom in the longitudinal direction (25).

The guide bar (82) in the exemplary embodiment has a cylindrical region (83), from which the solid prism (103), which is e.g. likewise cylindrical, protrudes centrally. The transition between the cylindrical region (83) and the solid prism (103) in the form of a cylindrical bar is formed by e.g. a circumferential supporting collar (84). The supporting collar (84) can be configured in the same way as the retaining collar (34). The cross-sectional area of the solid prism (103) is geometrically similar to the inner cross-sectional area of the hollow prism (102), such that the hollow prism (102) can accommodate and guide the solid prism (103). In the exemplary embodiment, the solid prism (103) has a locking pin (105), which is e.g. spring-loaded and which engages in the longitudinal slot (104) of the hollow prism (102) in the event that a sliding joint (101) is installed. Thus, e.g. a means of both torsion protection and pull-out protection for the sliding joint (101) is formed. Other configurations of torsion protection and pull-out protection means are also possible.

The pressing body (51) can also be configured as a roller in this exemplary embodiment. The roller radius corresponds e.g. to the above-mentioned radius of the pressing surface (57). The pressing body (51) in this case is mounted e.g. on both end faces rotatably on a fork-shaped guide bar (82).

The restoring device (111) in this exemplary embodiment also comprises a spring energy store (115) in the form of a compression spring (115). This is constructed e.g. as described in connection with the first exemplary embodiment. The compression spring (115) can, for example, be fixed in a form-fitting manner on the supporting collar (84) and on the retaining collar (34). For example, the applicator (10) can then be executed without additional pull-out protection means.

Instead of a cylindrical coil compression spring, the spring energy store (115) can also be in the form of a disc spring assembly, a conical coil compression spring, etc. The sliding joint (101) can have e.g. a haptic indicator (116). For example, the sliding joint (101) is formed such that, shortly before reaching the usage position (12), a latching lug has to be overcome. The resistance, which can be felt by the operator, briefly becomes higher while the latching lug is being overcome, and then suddenly drops again. The latching lug can have differently configured flanks, such that the operator receives a different haptic signal if the pressing force is accidentally reduced. A visual or acoustic indicator (116) is also possible.

When applying the applicator (10) illustrated in FIG. 9, the operator draws the applicator (10) over the microneedle patch (130) after removing the cover film (136). While doing so, he presses the applicator (10) on to the microneedle patch (130). The applicator (10) can be held vertically or obliquely, with the handle piece (21) being inclined e.g. towards the patch's longitudinal direction (135) in the event of an oblique position. The applicator (10) is then drawn. The compression spring (115) is compressed. By means of the e.g. haptic signal, the operator is informed whether the pressing force is sufficient or must be increased.

FIGS. 10-12 show an applicator (10) with an integrated dispensing device (121). The handle piece (21) of this applicator (10) is in the form of a housing (35), in which the dispensing device (121) for a microneedle patch (130) and a pressing body group (50) are arranged.

The dispensing device (121) comprises e.g. six cylindrical bearing rollers (122-125). For example, two of these bearing rollers (122, 123) are rotatably mounted above the cover film (136) of a microneedle patch (130) and e.g. four bearing rollers (124, 125) below this microneedle patch (130) in the housing (35). The microneedles (132) of the microneedle patch (130) point downwards. The individual bearing rollers (122-125) can also be connected to each other by means of a linkage. The mutually opposite bearing rollers (122, 124; 123, 125) above and below the microneedle patch (130) can also be loaded against each other e.g. by means of a tension spring. The axes of rotation (126) of all the bearing rollers (122-125) are aligned parallel to one another. The upper bearing rollers (122, 123) are e.g. formed continuously. Their roller length oriented transverse to the patch's longitudinal direction (135) is greater than or equal to the width of the microneedle patch (130). It is also possible, however, to replace each of these upper bearing rollers (122; 123) with two individual rollers, whose roller length is e.g. shorter than half the patch width.

The lower bearing rollers (124, 125) are short rollers (124, 125). Their length in the patch's transverse direction corresponds e.g. to the width of the lateral projection (138) of the overpatch (133) beyond the microneedles (132). The bearing rollers (122-125) can be rotatable freely or in a controlled manner. The normal force of the aforementioned tension spring can also increase the roll resistance or rolling resistance of the bearing rollers (122-125).

The microneedle patch (130) is e.g. substantially configured as described in connection with the preceding exemplary embodiments. In the housing (35), the patch's longitudinal direction (135) forms an angle for example of between 30 degrees and 60 degrees, e.g. 45 degrees, with the longitudinal direction (25) of the handle piece (21). The cover film (136) lies on the microneedle patch (130) e.g. such that it adheres over the entire surface. However, the cover film (136) can also be connected to the microneedle patch (130) as described in connection with the exemplary embodiment of FIGS. 5-8. At the end facing the pressing body (51), the overpatch (133) and the cover film (136) are separated from one another in the illustration of FIG. 10.

The pressing body group (50) is pivotably mounted in the housing (35) in a pivot joint (91). In the exemplary embodiment, the pivot axis (95) of the pivot joint (91) is arranged in the axis of rotation (126) of the first upper bearing roller (122). The pressing body group (50) comprises a pressing body (51) and a fork-shaped lever arm (85). This lever arm (85) is mounted in the pivot bearing (91). In the exemplary embodiment, the fork (86) of the lever arm (85) carries an axle rod (87), on which the pressing body (51) in the form of a cylindrical roller is rotatably mounted. The radius of the roller (51) corresponds e.g. to the radius of the roller-shaped pressing body (51) described in connection with the exemplary embodiment of FIG. 9. However, it is also possible to connect the pressing body (51) rigidly to the lever arm (85). For example, the enveloping contour of the pressing body (51) in this case can correspond to the enveloping contour of one of the non-rotatable pressing bodies (51) described in connection with the aforementioned exemplary embodiments.

The applicator (10) has an internal restoring device (111) arranged in the housing (35). This comprises a spring energy store (115), which is supported on the housing (35) and on a spring plate (117) which is configured in the manner of a yoke. The yoke arms (118) of the spring plate (117) engage around the pressing body (51) at the end faces (69) thereof. They are connected to the axle rod (87) of the pressing body (51). For example, they are pivotably mounted on the axle rod (87).

The spring energy store (115) in this exemplary embodiment is in the form of a compression spring (115). However, it is also possible for the spring energy store (115) to be in the form of a disc spring assembly, a tension spring, etc.

In the illustrations of FIGS. 10-12, a pointer arm (119) with an indicator (116) is arranged on the spring plate (117). The housing (35) has e.g. a viewing window (36). The indicator (116) is not visible therethrough in the illustration of FIG. 10. In the illustrations of FIGS. 11 and 12, which show the loaded applicator (10), the indicator (116) is visible through the viewing window (36).

On the housing (35), a retaining hook (37) is furthermore secured. This penetrates and engages behind the spring plate (117). This retaining hook (37) forms a means of securing the restoring device (111) against being lifted off.

The base (38) of the housing (35) has an opening (39), through which the overpatch (133) protrudes in the illustration of FIG. 10. It is also possible that the opening (39) is closed off in the shipping position of the applicator (10). The microneedle patch (130) in this case is moved to the ready position (13) illustrated in FIG. 10 e.g. by means of a slider, handle, etc. that can be actuated from outside the housing (35).

A guide wedge (42) is arranged on the base (38) between the opening (39) and the pressing body (51) in the housing interior (41). This guide wedge (42) has a separating edge (43) and a guiding surface (44) bordering the separating edge (43), oriented towards the housing interior (41). The guiding surface (44) can be configured e.g. in the same way as the upper guiding surface (65) of the exemplary embodiment illustrated in FIGS. 5-8.

When a pressing body (51) according to FIG. 6 is employed in an applicator (10) according to FIG. 10, the guide wedge (42) can be omitted. In the event of a form-fitting connection between microneedle patch (130) and cover film (136), the pressing body group (50) from the exemplary embodiment of FIGS. 5-8 can also replace the guide wedge (42) and the pressing body (51). The guide elements (71) in this case can also be arranged on the housing side.

In the ready position (13) illustrated in FIG. 10, the overpatch (133) abuts against the pressing surface (57). The cover film (136) is guided in part along the guiding surface (44). The pressing body (51) is in the idle position (11) relative to the housing (35). The restoring device (111) is free of load.

When the applicator (10) is pressed and drawn along the skin (1), the microneedle patch (130) is drawn out of the dispensing device (121), see FIG. 11. The pressing body (51) is pressed in towards the usage position (12), loading the restoring device (111). The indicator (116) becomes visible in the viewing window (36). By means of the pressing body (51), the microneedle patch (130) is pressed on to the patient's skin (1), pressing the microneedles (132) into the skin (1). The pressing body (51) rolls over the microneedle patch (130) during this operation. When the applicator (10) is drawn further in the drawing direction (15), all the microneedles (132) are pressed in. After the microneedle patch (130) has been rolled across completely, the microneedle patch (130) is fixed on the skin (1), see FIG. 12.

The above-mentioned exemplary embodiments can also be combined with one another.

1. An applicator having a handle piece and a pressing body for a microneedle patch,
wherein the pressing body is connected to the handle piece,
wherein
the pressing body is mounted for movement relative to the handle piece in a sliding joint or in a pivot joint between an idle position and a usage position by means of external operating forces and an internal restoring device,
wherein the pressing body has a pressing surface which is spaced apart from the pivot joint or sliding joint,
wherein in the idle position the pressing body is not loaded by external operating forces and the restoring device has the lowest internal energy, and
wherein the pressing body is movable relative to the handle piece towards the usage position by means of external operating forces acting on the pressing surface, the internal energy of the restoring device thus being increased.

2. The applicator according to claim 1, wherein
a securing element limits the range of movement in the idle position.

3. The applicator according to claim 1, wherein
the pressing body or a pressing body group containing the pressing body is mounted on or in the handle piece.

4. The applicator according to claim 1, wherein
the restoring device has a load-dependent visual, haptic or acoustic indicator.

5. The applicator according to claim 1, wherein
the pressing surface is a continuously curved surface, of which the at least one radius of curvature center line with the smallest radius is normal to a longitudinal direction and skew to a longitudinal axis of the handle piece at least in the usage position.

6. A unit comprising an applicator according to claim 1 and a microneedle patch having a plurality of microneedles,
wherein
the smallest radius of the pressing surface is at least one and a half times the product of the circle constant π and the length of a heel of the microneedle in the microneedle patch's longitudinal direction.

7. The unit according to claim 6, wherein
the microneedle patch carries a cover film at least in part and in that the applicator has a wedge-shaped separating edge with an adjacent guiding surface for guiding away the cover film.

8. The unit according to claim 7, wherein
the separating edge is formed on the pressing body.

9. The unit according to claim 8, wherein
guide elements are arranged on the pressing body, and wherein guide elements engage at least over an overpatch of the microneedle patch and over the cover film.
